SEQUENCE LISTING (3 sequences)

SEQ ID NO: 1 - DNA, 9 bp, Artificial Sequence
  modified protein initiation codon
gaaagatgt

SEQ ID NO: 2 - DNA, 31 bp, Artificial Sequence
  PCR primer
gatccccggggatccaaacatgagccgcctg

SEQ ID NO: 3 - DNA, 31 bp, Artificial Sequence
  PCR primer
gatccccggggttggactaaaagatcgcgag

(19) United States
(12) Patent Application Publication
(10) Pub. No.: US 2009/0238802 A1
(43) Pub. Date: Sep. 24, 2009

(54) Intraperitoneal Delivery Of Genetically Engineered Mesenchymal Stem Cells

(76) Inventors: Padmavathy Vanguri, Cockeysville, MD (US); Joseph D. Mosca, Ellicott City, MD (US)

Correspondence Address: MCANDREWS HELD & MALLOY, LTD, 500 WEST MADISON STREET, SUITE 3400, CHICAGO, IL, 60661, US

(21) Appl. No.: 12/360,913

(22) Filed: Jan. 28, 2009

Related U.S. Application Data
(62) Division of application No. 10/446,450, filed on May 28, 2003.
(60) Provisional application No. 60/384,759, filed on May 31, 2002.

(51) Int. Cl. A61K 35/12; A61P 3/00

(52) U.S. Cl. 424/93.7

(57) ABSTRACT

A method of expressing at least one protein in an animal by intraperitoneal administration of mesenchymal stem cells genetically engineered with at least one polynucleotide encoding the at least one protein. The method may be employed in treating lysosomal storage disorders, such as Fabry Disease, or arthritic disorders, or hemophilia, for example.

This application is a divisional of U.S. patent application Ser. No. 10/446,450, filed on May 28, 2003, which claims priority to U.S. Provisional Patent Application Ser. No. 60/384,759, filed on May 31, 2002, the contents of which are incorporated herein by reference in their entireties.

This invention relates to the expression of proteins in an animal through the administration of genetically engineered cells to the animal. More particularly, this invention relates to the expression of therapeutic proteins in an animal through the intraperitoneal administration of genetically engineered mesenchymal stem cells to the animal. Still more particularly, this invention relates to the treatment of lysosomal storage disorders such as, for example, Fabry Disease, Gaucher's Disease, Farber's Disease, Niemann-Pick Disease, Hurler-Schie syndrome, Hunter's Disease, Sanfillippo syndrome, Types A and B, beta-glucoronidase deficiency, Pompe's Disease, and von Gierke's Disease, through the intraperitoneal administration of mesenchymal stem cells genetically engineered with a polynucleotide encoding an agent for treating a lysosomal storage disorder.

This invention also relates to the treatment of other diseases that require the delivery of therapeutic proteins, such as, for example, clotting factors, cytokines, such as, but not limited to, G-CSF and GM-CSF, cytokine receptors, erythropoietin, or hormones, such as, but not limited to insulin, to multiple organs and/or the circulatory system.

Mesenchymal stem cells (MSCs) are pluripotent cells residing in bone marrow that give rise to multiple connective tissues such as bone marrow stroma, bone, cartilage ligament, tendon, muscle, and fat. Mesenchymal stem cells can be isolated and expanded ex vivo in the absence of added growth factors as a non-differentiated adult stem cell population. These cells retain their pluripotency and can be stimulated to differentiate down various mesenchymal lineages. Mesenchymal stem cells demonstrate immune privilege which is reflected in their poor recognition by naive T-cells. This is in part due to the absence of HLA class II or T-cell co-stimulatory molecules on their cell surface.

Mesenchymal stem cells also may be employed in gene therapy. Mesenchymal stem cells are transduced efficiently with retroviruses. Transduced mesenchymal stem cells retain the potential to differentiate and continue to express transgenes after differentiation.

One gene therapy application that employs genetically engineered mesenchymal stem cells is the administration of mesenchymal stem cells genetically engineered with an alpha-galactosidase A gene as a treatment of Fabry Disease. Fabry Disease is a lysosomal storage disorder, where the missing alpha-galactosidase A enzyme results in the pathologic accumulation of globotriaosylceramide lipids in the tissues.

Mice have been injected intramuscularly with mesenchymal stem cells genetically engineered with an alpha-galactosidase gene. Subsequent to the administration of the genetically engineered mesenchymal stem cells, the mice were evaluated for expression of alpha-galactosidase. Such evaluation showed that a significantly high level of alpha-galactosidase A was present in the injected muscles up to 4 weeks after administration of the genetically engineered mesenchymal stem cells; however, no increase in enzyme activity was seen in other organs, such as the liver, kidney, and spleen. Such results may be due to the receptor mediated uptake of enzyme by the surrounding muscle tissue which does not create a strong enough gradient for the enzyme to leave the muscle, enter the circulation, and reach other organs.

In accordance with an aspect of the present invention, there is provided a method of expressing a protein in an animal. The method comprises administering intraperitoneally to the animal mesenchymal stem cells genetically engineered with at least one polynucleotide encoding at least one protein. The mesenchymal stem cells are administered in an amount effective to express said at least one protein in an animal.

In a preferred embodiment, there is provided a method of treating a lysosomal storage disorder by administering intraperitoneally to an animal mesenchymal stem cells genetically engineered with at least one polynucleotide encoding an agent for treating a lysosomal storage disorder.

In another embodiment, there is provided a method of treating an arthritic disorder, including, but not limited to, rheumatoid arthritis and osteoarthritis, by administering intraperitoneally to an animal mesenchymal stem cells genetically engineered with at least one polynucleotide encoding an agent for treating an arthritic disorder.

In yet another embodiment, there is provided a method of treating hemophilia in an animal by administering intraperitoneally to an animal mesenchymal stem cells genetically engineered with at least one polynucleotide encoding a clotting factor.

In a further embodiment, there is provided a method of treating diabetes in an animal by administering intraperitoneally to an animal mesenchymal stem cells genetically engineered with a polynucleotide encoding insulin.

Although the scope of the present invention is not intended to be limited to any theoretical reasoning, it is believed that when genetically engineered mesenchymal stem cells are administered intraperitoneally, such mesenchymal stem cells have more direct access to many of the internal organs. In addition, the peritoneal wall is highly vascularized and proteins are absorbed very efficiently.

In one embodiment, the mesenchymal stem cells include a cell surface epitope (e.g., CD105) specifically bound by antibodies produced from hybridoma cell line SH2, deposited with the ATCC under accession number HB10743. The mesenchymal stem cells may further include a cell surface epitope (e.g., CD73) specifically bound by antibodies produced from hybridoma cell line SH3, deposited with the ATCC under accession number HB10744 or hybridoma cell line SH4, deposited with the ATCC under accession number HB10745.

The term “polynucleotide,” as used herein, means a polymeric form of nucleotide of any length and includes ribonucleotides and deoxyribonucleotides. Such term also includes single and double stranded DNA, as well as single and double stranded RNA. The term also includes modified polynucleotides such as methylated or capped polynucleotides.

In one embodiment, the mesenchymal stem cells are supported on a support, preferably a particulate or spherical support and more preferably a macroporous spherical support or macroporous bead. In general, the particles or spheres or beads have a size of from about 130 microns to about 380 microns. In one embodiment, the support is a macroporous gelatin bead. An example of macroporous gelatin beads which may be employed are sold under the name CultiSpher by Percell Biolytica (distributed by Hy Clone).

In another embodiment, the support may be a support which may be implanted intraperitoneally. Examples of such supports include, but are not limited to, polyglycolic acid (PGA), poly L-lactic acid (PLLA), alginate, and gelatin sponges, such as, for example, Gel Foam.

The at least one protein encoded by the at least one polynucleotide may be any protein known to those skilled in the art. Examples of proteins which may be encoded by the at least one polynucleotide include, but are not limited to, those described in U.S. Pat. No. 5,591,625.

In one embodiment, the at least one protein is an enzyme. Enzymes which may be encoded by the at least one polynucleotide include, but are not limited to, alpha-galactosidase A, glucosidase, ceramidase, sphingomyelinase, alpha-iduronidase, iduronate sulfatase, heparan-N-sulfatase, alpha-N-acetylglucosaminidase, beta-glucoronidase, alpha-glucosidase, and glucose-6-phosphatase. In one embodiment, the enzyme is alpha-galactosidase A.

The at least one polynucleotide may be introduced into the mesenchymal stem cells as a naked polynucleotide (DNA or RNA) sequence, or the at least one polynucleotide may be contained in an appropriate expression vector, such as a plasmid vector or a viral vector. When a viral vector is employed, the viral vector may be a DNA viral vector, such as an adenoviral vector, an adeno-associated virus vector, a Herpes virus vector, or a vaccinia virus vector, or the viral vector may be an RNA viral vector, such as a retroviral vector or a lentiviral vector.

In one embodiment, the at least one polynucleotide encoding a protein is contained in a retroviral vector, which is integrated into the mesenchymal stem cells by means known to those skilled in the art, such as, for example, by transduction employing a retroviral supernatant produced from transfected packaging cell lines.

The genetically engineered mesenchymal stem cells are administered intraperitoneally to the animal in an amount effective to express the at least one protein in the animal. The animal may be a mammal, including human and non-human primates. In general, the genetically engineered mesenchymal stem cells are administered in an amount of from about 1×10⁵ cells/kg to about 1×10⁸ cells/kg, preferably from about 1×10⁶ cells/kg to about 1×10⁷ cells/kg. The exact amount of mesenchymal stem cells to be administered is dependent on a variety of factors, including, but not limited to, the age, weight, and sex of the patient, the disease or disorder being treated, and the extent and severity thereof.

The present invention is applicable particularly to the treatment of lysosomal storage disorders, such as, but not limited to, Fabry Disease, Gaucher's Disease, Farber's Disease, Niemann-Pick Disease, Hurler-Schie syndrome, Hunter's Disease, Sanfillippo syndrome, Types A and B, beta-glucoronidase deficiency, Pompe's Disease, and von Gierke's Disease. Thus, the mesenchymal stem cells may be genetically engineered with at least one polynucleotide encoding a therapeutic agent for the treatment of a lysosomal storage disorder. Such therapeutic agents, include, but are not limited to, alpha-galactosidase A (for treating Fabry Disease), beta glucosidase (for treating Gaucher's Disease), ceramidase (for treating Farber's Disease), sphingomyelinase (for treating Niemann-Pick Disease), alpha-iduronidase (for treating Hurler-Schie syndrome), iduronate sulfatase (for treating Hunter's Disease), heparan-N-sulfatase (for treating Sanfillippo syndrome, Type A), alpha-N-acetylglucosaminidase (for treating Sanfillippo syndrome, Type B), beta-glucoronidase (for treating beta-glucoronidase deficiency), alpha-glucosidase (for treating Pompe's Disease), and glucose-6-phosphatase (for treating von Gierke's Disease).

In one embodiment, the present invention is employed in treating Fabry Disease. In one embodiment, a retroviral vector including an alpha-galactosidase A gene is transduced into mesenchymal stem cells. The transduced mesenchymal stem cells then are administered intraperitoneally to a patient, whereby alpha-galactosidase A is expressed by the genetically engineered mesenchymal stem cells in the patient.

The present invention also is applicable to treating an arthritic disorder, such as, but not limited to, rheumatoid arthritis and osteoarthritis. Thus, the mesenchymal stem cells may be genetically engineered with at least one polynucleotide encoding an agent for treating an arthritic disorder. Such agents include, but are not limited to, TNF receptors, including TNF-RII, and interleukin receptors and receptor antagonists, including the interleukin receptor, Interleukin 1-RII, and Interleukin-1 receptor antagonists.

In one embodiment, the present invention is employed in treating rheumatoid arthritis. In one embodiment, a retroviral vector including a soluble TNF-RII gene is transduced into mesenchymal stem cells. The transduced mesenchymal stem cells then are administered intraperitoneally to a patient, whereby soluble TNF-RII is expressed by the genetically engineered mesenchymal stem cells in the patient.

The present invention also is applicable to the treatment of hemophilia. Thus, the mesenchymal stem cells may be genetically engineered with a polynucleotide encoding a clotting factor. Such clotting factors include, but are not limited to, Factor VIII and Factor IX. The mesenchymal stem cells then are administered intraperitoneally to a patient, whereby the clotting factor is expressed by the genetically engineered mesenchymal stem cells in the patient.

The present invention also is applicable to the treatment of diabetes. Thus, mesenchymal stem cells may be genetically engineered with a polynucleotide encoding insulin. The genetically engineered mesenchymal stem cells then are administered intraperitoneally to a patient whereby insulin is expressed by the genetically engineered mesenchymal stem cells in the patient.

### BRIEF DESCRIPTION OF THE DRAWINGS

The invention now will be described with respect to the drawings, wherein:

FIGS. 1A and 1B are graphs of αGalA activity in the muscles of Fabry knockout mice at 14 and 28 days, respectively, after intramuscular injection of human mesenchymal stem cells (MSCs) transduced with an αGalA gene;

FIGS. 2A, 2B, and 2C are graphs showing the amount of αGalA in the livers, kidneys, and spleens, respectively, of knockout mice that were given intraperitoneal injections of human MSCs transduced with an αGalA gene;

FIG. 3 shows the attachment of MSCs transduced with an αGalA gene to Cultisphers;

FIG. 4 shows graphs showing αGalA enzyme activity in livers and kidneys of mice at 14 days after intraperitoneal administration of human MSCs transduced with an aGalA gene;

FIG. 5 is a graph showing Gb3 lipid levels in mice that were given intraperitoneal injections of human MSCs transduced with an αGalA gene;

FIG. 6 is a graph showing Gb3 lipid levels in the livers of knockout mice that were given intraperitoneal injections of human MSCs transduced with an αGalA gene;

FIG. 7 is a graph showing systemic levels of soluble TNFRII (sTNFRII) in Fisher rats that were given intraperitoneal or intramuscular injections of MSCs transduced with an sTNFRII gene;

FIG. 8 shows schematics of the vectors pOT24, pN2* neo, pJM538neo, and MGIN;

FIG. 9 is a graph showing levels of human Interleukin-3 (hIL-3) in the serum of mice implanted with ceramic cubes including human MSCs transduced with the vector pJM538neo; and

FIG. 10 shows cross-sections of empty ceramic cubes and ceramic cubes which contained human mesenchymal stem cells transduced with the hIL-3 gene.

The invention now will be described with respect to the following examples; however, the scope of the present invention is not intended to be limited thereby.

### EXAMPLE 1

### Materials & Reagents

Protamine Sulfate (Sigma)
Research grade VSV-G-pseudotyped α-galactosidase A retroviral supernatant using clinical αGalA vector (pOT312)
D-PBS (Gibco BRL cat. no. 14190-136, C04006)
Trypsin-EDTA (Gibco BRL cat. no. 25300-054, C20009)

Fetal Bovine Serum (Hyclone cat. no. SH30071.03, C06007)
Cryoserv-DMSO(C03004)

Primary human mesenchymal stem cells (Donors 475 and 532) Human MSCs from donor hMSC 475/p3 or p4 and 532/p3 which have been transduced with αGalA retrovirus.
Rat mesenchymal stem cells and rat MSC culture medium
Human MSC Culture Media
Phenol red free, serum free DMEM (SFM)
T-80 Tissue Culture Flasks (Nunc cat. no. 178891)
T-185 Tissue Culture Flasks (Nunc DA21580)
Two stacks and Ten-stacks (Nunc).
4-methylumbelliferyl-α-D galactopyranoside, (Research Product International, No. M65400)
N-acetyl-D galactosamine (Sigma, No. A-2795)
4-methylumbelliferyl-2 acetamido-2-Deoxy-β-D-glucopyranoside
(Research Product International, No. M64100)
4-Methylumbelliferone (Sigma, No. M-1381) Citric Acid (Fisher Scientific, No. A940-500)
Sodium Phosphate, Dibasic salt (Sigma, No. S-7907)
Bovine Serum Albumin (Gibco BRL, No. 11018-025)
Taurocholic Acid, Sodium Salt (Sigma, No. T-9034)
Reagents for lipid extraction, HPLC
CultiSpher-G (HyClone, DG-0001-00)
BCA-Protein Kit (Pierce, Rockford Ill.)

### Mice:

Fabry Knock-Out mice were obtained from NIH and bread at UMBI animal core facility. Mice were 20 weeks old for Intra-muscular injection and 16-weeks old for the Intra-peritoneal experiments.

Wild Type control mice C57BI6/129 from Jackson Laboratories Mice will be age-matched to the KO-mice and will be used when they are 16-weeks old.

### Equipment:

Incubator (37° C., 5% CO₂ & 90% humidity)
Beckman GS6-R Centrifuge
Sonic Dismembrator (Fisher Scientific, model F550)
Eppendorf Centrifuge 5415C (Brinkman Instruments, No. 2236527-4)
FMAX, Plate Reader (Molecular Devices, LabSystems RS-232-C)
ThermoMax Microplate Reader (Molecular Devices, LabSystems RS-232-C)
Sonic Dismembrator (Fisher Scientific, model F550)
Tissue Grinders (Kendall Precision Disposable)

### Methods

Preparation of VSV-G peudotyped retroviral supernatant: A retroviral vector containing human αGalA was constructed using the pBA-9b retroviral back bone (Sheridan et al., Mol. Ther., 2000, 2:262-275). VSV-G pseudotyped retrovirus was produced in the human 293 (2-3) packaging cell line (Sheridan et al., Mol. Ther. 2000, 2:262-275). The virus was concentrated 30 fold and frozen at −80° C.

Transduction of MSCs: (Lee et al., Mol. Ther. 2001, 3:857-866)

Day 0: hMSCs (p.0) isolated and cryopreserved by Human Tissue Culture Core facility were thawed, counted and plated at a seeding density of 6.25×10³ cells/cm² (5×10⁵ cells/T-80 flask in 15 ml of hMSC media). Cells were cultured overnight at 37° C. in 5% CO₂ humidified incubator.

Days 1-5 (summary, procedure): After removing hMSC media from each T-80 flask the required amount of frozen concentrated α-Gal-A retroviral supernatant was thawed in a 37° C. water bath. Transductions were done as follows: 15 ml of 1:5 dilution of αGalA retroviral supernatant supplemented with Protamine Sulphate (15 μg/ml, Sigma) was added to the MSCs in T-80 flasks. T-80 flasks were centrifuged at 3,000 rpm (1,640×g) for 1 hour at room temperature (20-25° C.) in a Beckman GS6-R. 15 mLs of hMSCs culture media was added to each flask to dilute the retroviral supernatant. Cells were cultured overnight (16-18 hours) at 37° C./5% CO₂/90% humidity. The centrifugal transduction was repeated the following day with fresh virus.

On day 3, Media-retroviral supernatant mixture was removed from all the flasks, and 15 ml of fresh hMSC media were added to each flask. hMSCs were cultured to confluency (p1). Cell cultures were examined visually. Once cultures were confluent, hMSCs were trypsinized and expanded through T185 cm² flasks or Two stack (p2) and finally in a Ten-stack (p3). Cultures were maintained at 37° C./5% CO₂/90% humidity by replacing with fresh medium every 3 days. At different passages, once cultures were between 90 and 100% confluent, culture media were removed and replaced with fresh hMSC media. Cells were incubated for 24 hours and aliquots of the culture supernatant were collected. Supernatants were filtered through a 0.45 μm filter and stored at −80° C. An α-Gal-A extracellular enzymatic activity assay was performed. Cells were harvested, and cell counts and viability were recorded. The cells were cryopreserved.

Control non-transduced MSCs were expanded to P3 similar to transduced cells except that they were not transduced with retrovirus.

Intramuscular delivery of αGalA-hMSCs: αGalA-hMSCs were thawed, washed and resuspended in phenol red free, serum free medium (SFM) at a concentration of 20×10⁶/ml. The mice were anesthetized with an IP injection of Nembutal. The lower back and hind limb fur were shaved. The skin was disinfected sequentially with alcohol, betadine and alcohol. A total of 200 μl of cell suspension containing 4×10⁶ cells was delivered to each mouse into both thighs using a tuberculin syringe. 100 μl of cell suspension were injected at 2 to 3 sites per leg into the belly of the thigh muscle as described below. Control mice received similar volume of SFM alone. Mice in groups 5-8 received intraperitoneal injections of Cyclosporine A (CsA) at a dose of 25 mg/Kg once a day for one week, starting at day −1 (day 0=day of cell implantation). They then received a dose of 20 mg/kg daily for an additional week.

Experimental design: Intra-muscular injection

| Group | Treatment | Time of Sac | # of mice |
|---|---|---|---|
| 1. | αGalA-MSCs | 2 wks | 5 |
| 2. | αGalA-MSCs | 4 wks | 5 |
| 3. | Vehicle | 2 wks | 5 |
| 4. | Vehicle | 4 wks | 5 |
| 5. | αGalA-MSCs + CsA | 2 wks | 5 |
| 6. | αGalA-MSCs + CsA | 4 wks | 5 |
| 7. | Vehicle + CsA | 2 wks | 5 |
| 8. | Vehicle + CsA | 4 wks | 5 |

Intraperitoneal Delivery of αGalA Transduced hMSC to Fabry KO Mice:

Transduced cells were thawed, washed and resuspended in hMSC medium. Required number of αGalA-transduced hMSC were prepared for delivery to Fabry KO mice according to the experimental design shown below.

Pilot Experiment: 25 mg Cultisphers

| Group/Mouse | Treatment | #of Cells | # of mice |
|---|---|---|---|
| 1. KO | αGalA-MSCs on CultiSphers | 5 × 10⁶ | 2 |
| 2. KO | αGalA-MSCs alone | 5 × 10⁶ | 2 |

Experiment 1: 5 mg Cultisphers

| Group/Mouse | Treatment | # of Cells | # of mice |
|---|---|---|---|
| 1. KO | αGalA-MSCs on CultiSphers | 4 × 10⁶ | 4 |
| 2. KO | αGalA-MSCs alone | 4 × 10⁶ | 4 |
| 3. KO | Control MSCs on Cultisphers | 4 × 10⁶ | 4 |
| 4. KO | Control MSCs alone | 4 × 10⁶ | 4 |
| 5. KO | Enzyme Supernatant from | — | 4 |
| | αGalA-hMSCs | | |
| 6. KO | SFM Alone | — | 4 |

CultiSpher-G beads were hydrated in Mg-free and Ca-free PBS at 10 mg/ml concentration for 1 hour and then autoclaved at 121° F. for 20 minutes. Cooled beads in solution were stored at 4° C.

For the pilot experiment, the required amount of beads and cells for two mice were incubated in one tube. Briefly, 50 mg of hydrated beads were centrifuged, the medium was removed and the beads were incubated with 10×10⁶ αGalA-hMSCs in 2 ml of hMSC medium.

In experiment-1, the loading of beads was performed in a separate tube for each mouse. For groups 1 and 3, 0.5 ml of beads containing 5 mg beads was pipetted into a 6 ml Falcon polypropylene tube. The tubes were centrifuged at 1500 rpm for 5 minutes and the medium was removed. The CultiSpher pellet was resuspended with 1 ml of SFM containing 4×10⁶ αGalA-hMSCs or control MSCs. The bead-cell suspensions were incubated at 37° C./5% CO₂ for 2 hours with gentle agitation every 15 minutes or on a horizontal roller table at the lowest speed. Beads and attached cells were allowed to settle for 3-5 minutes and rinsed two times, allowing the beads to settle each time in between washes. Finally the beads and cells were suspended in 0.5 to 0.6 ml of SFM.

Mice in groups 1 and 3 received intraperitoneal injections of the cell/bead suspension. Groups 2 and 4 received 4×10⁶ αGalA-hMSCs or control MSCs suspended in 0.5 ml of SFM without any beads.

For group 5, 4×10⁶ αGalA-hMSCs were loaded on 5 mg beads as for group 1, one day before injection of mice. After washing, the beads/cells were placed in a 24 well plate with 0.6 ml of SFM and incubated overnight. After 24 h the medium was removed from the beads and injected into the mice. An aliquot of the medium was used to measure enzyme activity released into the medium. Group 6 received SFM alone.

For injection of mice, the slurry of beads and cells was drawn into a 1 ml syringe fitted with a 20 gauge needle. The mice were injected intraperitoneally, making sure that all the beads/cells and the medium were injected.

### Sacrifices and Tissue Harvest

### Immunohistochemistry, αGalA Enzyme Assay and Gb3 Lipid Analysis:

At the required time points post-injection, animals were sacrificed by CO₂ inhalation according to approved animal protocols. Wild type age matched controls were also sacrificed and organs collected for enzyme and lipid analysis.

Blood was collected by cardiac puncture from all the groups. At necropsy, tissues were harvested and split into three parts for: 1) αGalA enzyme activity, 2) lipid extraction and 3) Histology. The organs harvested included liver, kidney, spleen, heart, brain, small intestine and lung. For the intramuscular experiment the thigh muscle was harvested as well.

For histology, a portion of each organ and one of the injected thigh muscles from each mouse was put into 10% neutral buffered Formalin. The tissues were then embedded in paraffin and cut into sections for immunohistochemistry of αGaIA. The sections were stained with anti-αGalA polyclonal antibody and detected with anti-rabbit biotin labeled antibody followed by streptavidin peroxidase. Positive staining was visualized with Diaminobenzidine (DAB)

For αGalA enzyme activity and lipid analysis the tissues were rinsed in PBS and frozen at −80 C. The tissues were weighed rapidly, and homogenized in buffer (28 mM citric acid/44 Mm disodium phosphate containing 3 mg/ml Sodium Taurocholate) at 100 or 200 mg/ml concentration using tissue grinders. The homogenate was then sonicated using a sonic dismembrator with 2 pulses for 20 and 10 sec each. A small aliquot was taken for protein quantitation. 400 μl of the homogenate was frozen away for lipid analysis. The rest of the homogenate was centrifuged in a microcentrifuge at maximum speed for 30 min. The supernatant was removed and centrifuged again for 10 min. The resulting supernatant was the tissue lysate. Again an aliquot of the lysate was taken for protein quantitation. αGalA enzyme activity of the lysates was measured with 5 mM 4-methylumbelliferyl αD-galactopyranoside with 0.1 M N-acetyl-D-galactosamine used as an inhibitor of α-N-acetylgalactosaminidase as described (Kusiak et al., J. Biol. Chem. 1978, 253:184-190 and Schiffmann et al., Proc. Natl. Acad. Sci. USA, 2000, 97:365-370).

Glycosphingolipids were isolated and HPLC analyses of Gb3 levels in organs was measured as described in (Schiffmann et al., Proc. Natl. Acad. Sci. USA, 2000, 97:365-370). The protein concentration of the homogenates and the lysates were analyzed using the BCA kit from Pierce Biochemical.

### Results:

### αGalA Enzyme Activity of the Various Organs:

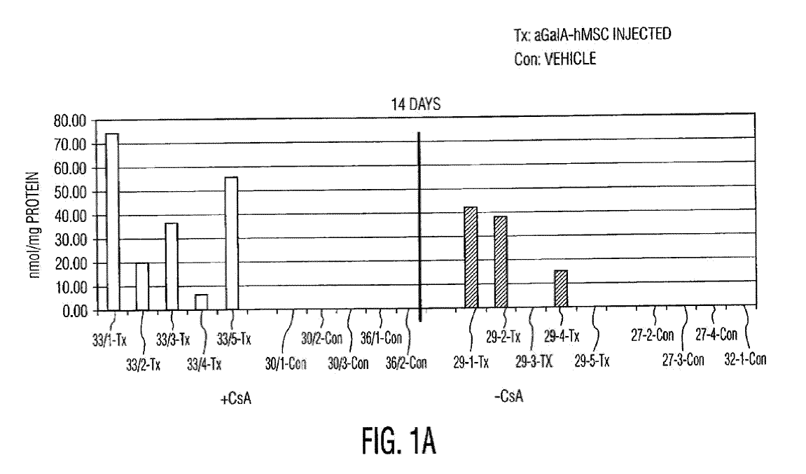

Intra-muscular injection of MSCs: Fabry KO-mice were injected intra-muscularly with αGalA-hMSCs or SFM. The amount of enzyme secreted by these MSCs, donor 475 p3 was estimated to be about 1000 nmole/h/1×10⁶ cells. The thigh muscles and organs were harvested and processed for measuring enzyme activity or for immunohistochemistry. As seen in FIGS. 1a,b, the muscles injected with αGalA-hMSCs^{(TX)} contained significant levels of αGalA enzyme activity at 14d and 28 d, while the vehicle^{(con)} injected muscles had almost no enzyme activity. Each bar represents muscle from an individual mouse. Also, irrespective of whether the mice received the immuno-suppressive agent CsA, the MSC-injected muscles contained high levels of αGalA enzyme activity at both time points. However there was no elevation in αGalA activity in the livers or kidneys of mice injected with αGalA-hMSCs (Table 1).

TABLE 1
αGalA Activity in Organs of Fabry KO mice: 14 days
after IM injection of αGalA-Transduced hMSCs

| | αGalA-Tx | Vehicle-Con |
|---|---|---|
| | nmole/mg | nmole/mg |
| Kidney + CsA | 0.12 +/− 0.0 | 0.15 +/− 0.05 |
| Kidney − CsA | 0.11 +/− 0.03 | 0.11 +/− 0.08 |
| Liver + CsA | 0.66 +/− 0.16 | 0.64 +/− 0.07 |
| Liver − CsA | 0.4 +/− 0.04 | 0.37 +/− 0.08 |
| Spleen + CsA | 3.10 +/− 0.76 | 2.7 +/− 0.53 |
| Spleen − CsA | 4.43 +− 1.58 | 2.27 +/− 0.56 |

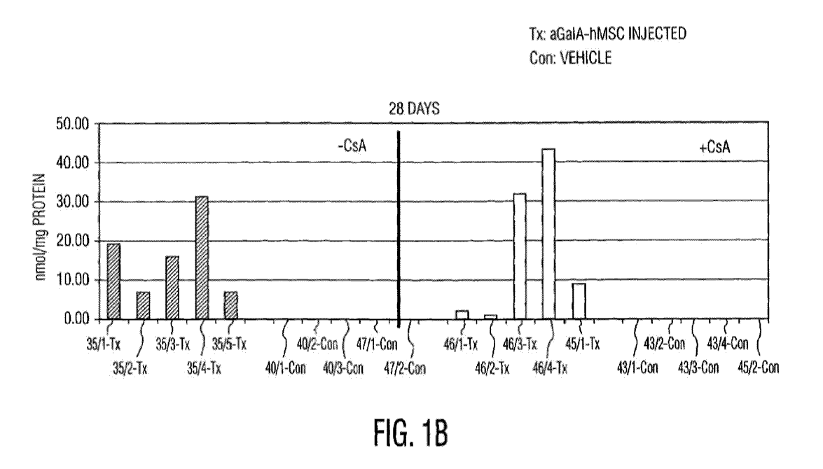

Intraperitoneal injection of MSCs: Fabry KO-mice were injected with αGalA-hMSCs attached to CultiSpher beads by IP injection. The transduced MSCs, donor 532-p3 were estimated to secrete about 2000 mmoles/h/1×10⁶ cells. Controls included beads alone, vehicle (SFM) or non-transduced MSCs +/−CultiSphers. In addition a group of mice were injected with enzyme supernatant from 4×10⁶ αGalA-hMSCs attached to beads. The enzyme activity of the supernatant was estimated to be 1385 nmoles/ml. The mice were harvested two weeks following the injection. The tissues from all mice were homogenized and the αGalA enzyme activity of the lysates was measured and expressed per mg protein. In the pilot experiment, the data showed that the maximum increase in enzyme activity was seen in the liver (FIG. 2). The KO mice have negligible αGalA enzyme in their tissues. On average a 6.5 fold increase in αGalA was seen in the livers of mice that received αGalA-hMSCs attached to CultiSphers when compared to mice that received CultiSphers alone (each bar represents a different mouse). The kidneys also showed an increase on average of 1.6 fold and the spleens showed an increase of 1.9 fold.

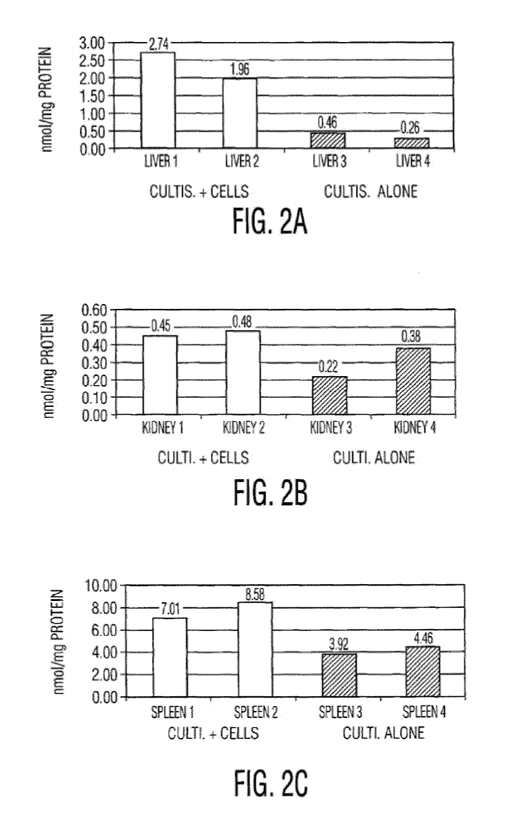

In the pilot experiment we used 25 mg of CultiSphers per mouse and we determined later with in vitro loading experiments that we could deliver similar number of cells using five fold less beads. FIG. 3 shows representative images of a CultiSpher bead taken from incubations of 5 mg beads with 1.25 to 10×10⁶ cells/ml. The beads were stained with (MTT) to visualize the presence of live MSCs attached to the beads. By increasing the MSC concentration in the incubation using the same number of beads we were able to attach more MSCs per bead. Thus, using 5 mg of beads made the consistency of the beads/cells easier to inject intraperitoneally. In addition, we found that mg of beads did not cause clumps of cells and beads to attach to the organs.

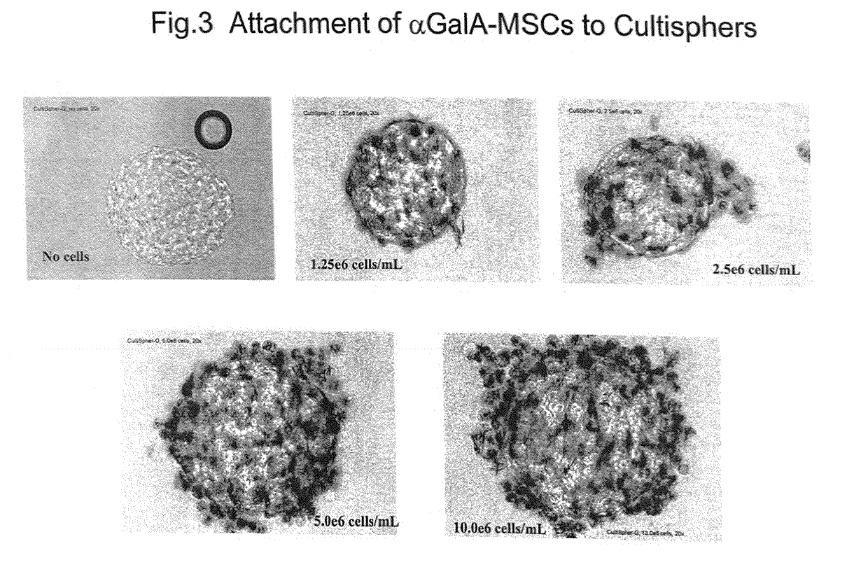

In experiment 1 we injected mice with 4×10⁶ cells loaded on 5 mg beads. In parallel loading experiments, it was determined that only 50-75% of the 4×10⁶ MSCs attached to the beads, thereby reducing the effective dose of MSCs delivered to the mice on the CultiSphers. The enzyme results are shown in FIG. 4. Once again we saw a dramatic mean increase of 4.2 fold in αGalA in the livers of mice that received αGalA-attached to Cultisphers. When mice were injected with αGalA hMSCs alone there was a 1.9 fold increase in liver αGalA. The enzyme supernatant containing αGalA secreted in the course of 24 h by 4×10⁶ αGalA-hMSCs attached to beads, had no effect on increasing the liver enzyme. Non-transduced (mock) MSCs had no effect whether they were attached to beads or not. The kidneys also showed an increase in enzyme activity when mice were injected with αGalA-hMSCs attached to beads. Similar results were seen with the spleen (not shown). The brain and hearts did not show any appreciable increase in enzyme activity (not shown).

Age-matched wild type mice were also analyzed for enzyme activity. The livers of normal wild type mice contained on average of 43 nmol/mg, the kidneys had 21 nmoles/mg and the spleens had a wide range with a mean of 156 nmol/mg of αGalA enzyme activity.

### Gb3 Lipid Analysis of KO-Mice Tissues:

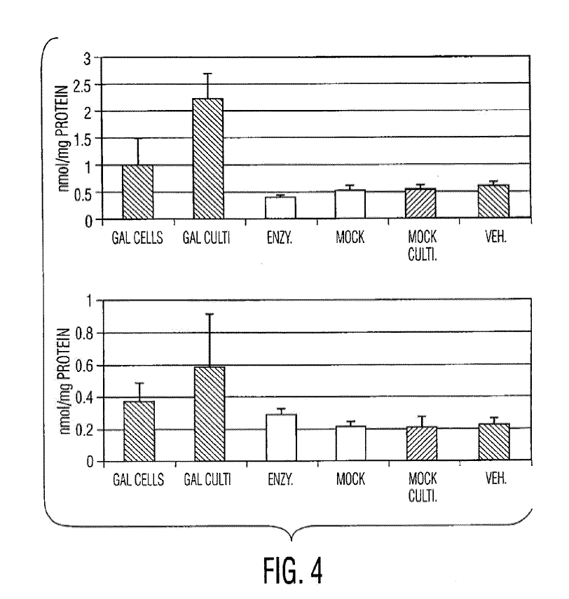

Glycosphingolipids were extracted from the organs of mice in the IP experiments. The level of Gb3 per mg protein was quantitated using HPLC. FIG. 5 shows the Gb3 levels (nmol/mg protein) in the livers of the mice from the pilot experiment. Mice that received αGalA-hMSCs+CultiSphers showed an average 67% decrease in the Gb3 levels of liver when compared to the mice that received cultisphers alone. Levels of individual mice are shown.

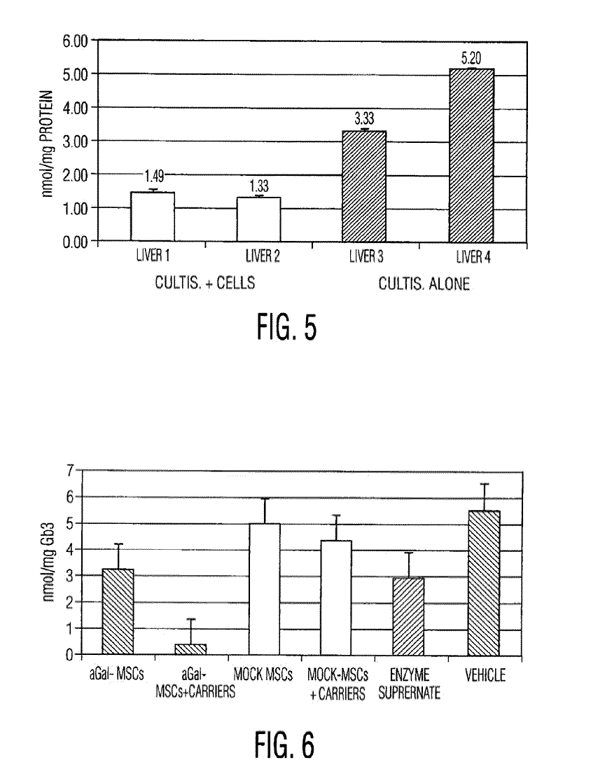

In experiment 1 we analyzed the Gb3 levels in the livers 14 days after injection. FIG. 6 confirmed data from the pilot experiment showing a dramatic, mean reduction by 90% in the Gb3 levels of livers of mice treated with αGalA-hMSCs+CultiSphers (MSCs+Carriers). Corresponding to the αGalA enzyme increase seen in FIG. 4, the mice that received αGalA-MSCs alone also showed a reduction in Gb3 in the liver, although not as much as with αGalA-MSCs-CultiSphers. The enzyme supernatant also caused a minimal reduction in Gb3. Gb3 levels also were reduced in the kidneys of mice treated with αGalA-hMSCs+CultiSphers (not shown).

The Gb3 levels of livers from wild type mice were a negligible 0.02 nmol/mg.

### EXAMPLE 2

### Intraperitoneal Delivery of Soluble TNFR-II Using Transduced MSCs

sTNFRII (extracellular portion of the type II TNF (p75) receptor) has been shown to be beneficial for rheumatoid arthritis by inhibiting the activity of TNF. Recombinant huTNFR:Fc was shown to both protect and prevent type-II collagen induced arthritis in mice when given as daily intraperitoneal injections (Wooley, et al J. Immunol. 151: 6602-6607, 1993). huTNFR:Fc is a dimeric fusion protein of the extracellular portion of p75 TNFR linked to the Fc portion of human IgG1.

The delivery of sTNFRII via gene-modified MSCs is investigated in this example. The extracellular portion of rat TNFRII (sTNFRII) was cloned in pJM573Neo, which is a Moloney Murine Leukemia Virus retroviral vector. (Mosca, et al., Clin. Orthop. Related Res., Vol. 379S, pgs. S71-S90 (2000). The gene was cloned as a fusion protein with the Fc portion of rat IgG along with an IRES-Neo^{r} gene for selection. Amphotropic retrovirus was produced in an AM-12 packaging cell line. The virus was used to transduce rat MSCs isolated from Fisher rats. The transduced rat MSCs were selected with Neomycin and expanded. The cells secreted sTNFRII into the medium. sTNFRII was measured with an ELISA kit from R & D Systems for detecting mouse sTNFRII.

Systemic delivery of sTNFRII via transduced rMSCs was evaluated in Fisher rats. MSCs were delivered either by intramuscular injection (IM) or by intra-peritoneal injection (IP). For IP delivery, MSCs were either injected as a suspension in serum free-medium or after attaching to Cultisphers as described for the alpha-GalA-transduced MSCs.

4 million transduced MSCs were injected IP (IP-cells+culti or IP-cells) or 2 million per thigh muscle at a total of 4 million per rat were injected IM (IM-cells). Each experimental group consisted of 6 rats. Control rats received non-transduced MSCs (Mock) by IM or IP injections. Each control group consisted of 4 rats. Rats were bled prior to injection of MSCs for baseline values and on days 4, 11, 18 and 28. The plasma was collected and frozen. sTNFRII levels were measured by ELISA.

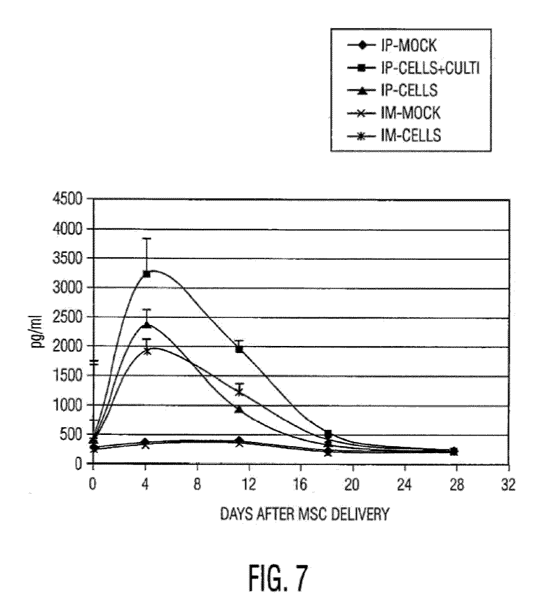

The results, as shown in FIG. 7, showed that rats that received sTNFRII-transduced MSCs had high levels of sTNFRII in their blood at day 4 that declined, but stayed significantly elevated by day 11, then further declined to appreciable levels by 18 days. Finally the levels were reduced to almost baseline levels by 28 days. Comparison of the different routes showed that transduced MSCs delivered IP after attachment to Cultisphers (IP-Cells+Culti) were the most effective and showed the highest range of sTNFRII levels in the blood for the longest time. MSCs given IP without attachment to Cultisphers (IP-cells) also delivered sTNFRII into the blood, although the levels comparatively were lower and also dropped down sooner than when cells were delivered on Cultisphers. IP delivery was more effective than IM (IM-cells). Non-transduced (Mock) MSCs did not increase sTNFRII above baseline in the blood whether given IM or IP.

Thus, mesenchymal stem cells genetically engineered with sTNRII are effective in the systemic delivery of sTNFRII when administered intraperitoneally. Such mesenchymal stem cells also may be genetically engineered with genes encoding other anti-arthritic agents, such as IL1-RII or IL-1 receptor antagonist, and be delivered intraperitoneally as well.

### EXAMPLE 3

### Materials and Methods

Isolation and culture expansion of hMSCs. Bone marrow samples were selected from healthy human donors (age 28-46 years) at the Johns Hopkins Oncology Center under an Institutional Review Board approved protocol. Human MSCs were isolated and cultured according to previously reported methods (Pittenger, et al., Human Cell Culture Series, Vol. 5, Chap. 10, pgs. 187-207 (2001). Briefly, heparinized bone marrow was fractioned over a 1.073 g/ml Percoll solution (Pharmacia Biotech, Piscataway, N.J.) and the mononuclear cells accumulated at the interface were plated in hMSC medium at a density of 3×10⁷ cells per 185 cm² in Nunclon Solo flasks (Nunc, Inc., Naperville, Ill.). Human MSC medium consisted of Dulbecco's modified Eagle's medium-low glucose (DMEM-LG) (Life Technologies, Gaithersburg, Md.) supplemented with 10% fetal bovine serum (FBS; Biocell Laboratories, Rancho Dominquez, Calif.) and 1% antibiotic-antimycoltic solution (Life Technologies). The FBS used in hMSC medium was selected based on its ability to maximize recovery and culture expansion of hMSCs from bone marrow that produce bone and cartilage in a ectopic implantation model (Lennon, et al., In Vitro Cell Dev. Biol., Vol. 32, pgs. 602-611 (1996)). Attached, well-spread hMSCs were visible at 5-7 days after initial plating and selectively accumulated and expanded by the removal of nonadherent and loosely attached cells during the medium changes. Confluent cultures were detached by trypsin-EDTA (Life Technologies) treatment and replated at 1×10⁶ cells per 185 cm² flask and denoted passage-1 cells.

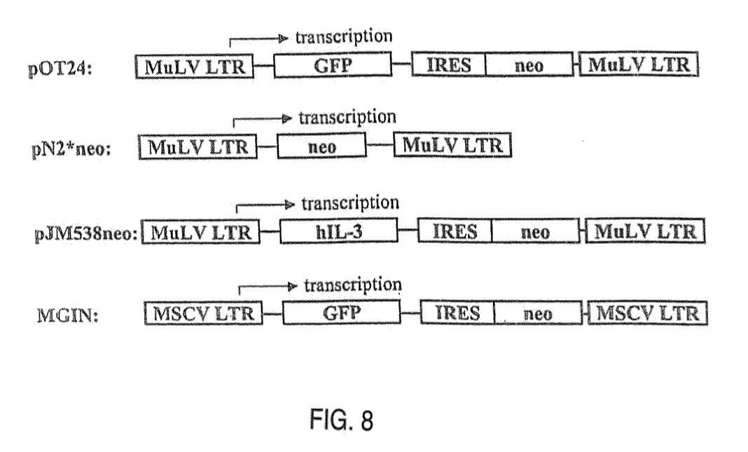

Retroviral vector construction and virus production. The schematic drawings of the vectors used in this report are presented in FIG. 8. The retroviral vector pOT24, expressing enhanced green fluorescent protein (GFP) of the jellyfish Aequorea Victoria, was constructed as described (Mosca, et al., Clin. Orthop. Relat. Res., Vol. 379S, pgs. S71-S90 (2000). The plasmid pN2*neo is a modification of the parent plasmid pN2 (Keller, et. al., Nature, Vol. 318, pgs. 149-154 (1985)), in which the protein initiation codon in the neomycin phosphotransferase gene was changed to GAAAGATGT (SEQ ID NO:1). The retroviral vector pJM538neo, expressing human interleukin 3 (hIL-3), was constructed by amplifying (using RT-PCR) the hIL-3 cDNA from human bone marrow RNA with synthetic oligonucleotides O-JM525 (5′ primer: 5′-GATCCCCGGGGATCCAAACATGAGCCGCCTG-3′) (SEQ ID NO:2) and O-JM526 (3′ primer: 5′-GATCCCCGGGbTTGGACTAAAAGATCGCGAG-3′) (SEQ ID NO:3), followed by cloning the fragment into the EcoRV site of pBluescript (Stratagene, La Jolla, Calif.). The hL-3 cDNA was transferred from the pBluescript vector to the retroviral vector pJM573neo (Mosca, 2000) using the BglII and XhoI sites, resulting in pJM538neo. The MGIN retroviral vector was construction as described by Cheng, et al. Gene Ther., Vol. 4, pgs. 1013-1022 (1997). In addition to specific transgenes transitonally regulated by the retroviral vector long terminal repeat, pOT24, pJM538neo, and MGIN contain the encephalomyocardifis virus internal ribosomal entry site (IRES) (Ghattas, et al., Mol. Cell. Biol., Vol. 11, pgs. 5848-5959 (1991)) for the additional translation of the neomycin phosphotransferase (neo) gene.

The retroviral vectors pOT24, pN2*neo, and pJM538neo were transfected into GP+E-86 ecotropic producer cells (Markowitz, et al., Adv. Exp. Med. Biol., Vol. 241, pgs. 3540 (1988)) (ATCC No. CRL-9642) and amphotropic retrovirus was prepared by transducing PA317 cells (Miller, et al., Mol. Cell. Biol., Vol. 6, pgs. 2895-2902 (1986)) (ATCC No. CRL-9078) twice with the ecotropic virus as described (Mosca, 2000). The retroviral vector MGIN was transiently cotransfected with VSVg envelope into φNX-GP producer cells (gift to Dr. Cheng from Dr. Gary Nolan, Stanford, Palo Alto, Calif.) (Kinsella, et al., Hum. Gene Ther., Vol. 7, 1405-1413 (1996)) using DOTAP (Boehringer Mannheim, Indianapolis, Ind.) and the procedure suggested by the manufacturer. The transfected cells were grown for 2 days and the retroviral supernatant was used to infect φNX-A. Populations of highly fluorescent cells were sorted by flow cytometry. Sorted cells were pooled and plated in 185-cm² flasks and the retrovirus-containing supernatant was collected as described (Mosca, 2000). Titers of pOT24, pN2*neo, pJM538neo, and MGIN-derived retroviruses were 1.2×10⁶, 6.4×10⁵, 1.0×10⁶, and 2-4×10⁵ colony-forming units/ml, respectively. All retrovirus supernatants were free of helper virus.

Retroviral transduction of hMSCs. Static and centrifugal procedures were used to optimize retroviral transduction of hMSCs. Transduction efficiency was assessed by two methods: neomycin-resistant colony formation and GFP fluorescence by flow cytometry analysis. For colony formation, hMSCs were transduced with the retroviral vector pN2*neo and for GFP fluorescence, hMSCs were transduced with the pOT24 retroviral vector (FIG. 8). These procedures established that centrifugation of hMSCs with retroviral supernatants at 1650 g for 1 h improved transduction efficiencies threefold. In addition to centrifugation, the retroviral packaging cell line used to package the retroviral vector enhanced gene transduction efficiencies. Human MSCs transduced with pOT24 by a one-cycle centrifugal transduction with retroviral preparations from the φNX retroviral packaging cell line resulted in 80% GFP-positive cells when fluorescence was measured by flow cytometry, compared to 40% GFP expression with retroviral preparations from the PA317 retroviral packaging cell line. These experiments led to the following procedure for retroviral transduction of hMSCs. Cells were plated in 80-cm² flasks (Nunc) at a density of 0.5×10⁶ cells in hMSC medium 24 h prior to retroviral transduction. The transduction cocktail consisted of retroviral supernatant and 8 μg/ml Polybrene (Sigma, St. Louis, Mo.); 15 ml of transduction cocktail was added to each flask and centrifuged for 1 h in a Beckman GS-6R centrifuge (Beckman Instruments, Palo Alto, Calif.) using microtiter plate carriers at 32° C. Two successive cycles of transduction further enhanced gene expression and were done routinely.

Flow cytometry analysis._Analysis of GFP fluorescence from hMSCs was performed by flow cytometry as previously reported (Majumbar, et al., J. Cell. Physiol., Vol. 176, pgs. 57-66 (1998)). Briefly, medium was removed from flasks, cell layers were washed twice with DPBS, and cells were detached by incubation with 0.25% trypsin-EDTA. Human MSCs were recovered by centrifugation and washed in flow cytometry buffer consisting of 2% BSA (Sigma) and 0.1% sodium azide (Sigma) in DPBS. Cells were collected by centrifugation and resuspended in flow cytometry buffer containing 1% paraformaldehyde (Electron Microscopy Sciences, Fort Washington, Pa.) immediately before being analyzed. Non-specific fluorescence was determined using hMSCs that were not transduced. Samples were analyzed by collecting 10,000 events on a Becton-Dickinson Vantage Instrument using Cell-Quest software (Becton-Dickinson).

Human MSCs Implantation into NOD/SCID mice and detection of hIL-3. Human IL-3 transduced hMSCs were G418 selected and implanted into NOD-SCID mice (The Jackson Laboratory, Bar Harbor, Me.). Cells were delivered unattached by intravenous, subcutaneous, and intraperitoneal routes or attached to matrices and implanted subcutaneously or intraperitoneally. For the latter, hMSCs were seeded on human fibronectin-coated porous hydroxyapatite/tricalcium phosphate (HA/TCP; 65% HA and 35% TCP& Zimmer, Warsaw, Ind.) 3-mm ceramic cubes and surgically implanted 5×10⁵ cells/cube, 10 cubes/animal). For intraperitoneal delivery, cells (5×10⁶) were attached to GelFoam (gelatin sponge derived from porcine skin; Pharmacia & Upjohn, Inc., Kalamazoo, Mich.), alginate disks (Keltone LCVR; Kelco Corp., San Diego Calif.; 2×10-mm diameter), or CultiSpher G beads (DG-0001-00; HyClone, Logan, Utah). Whereas the unattached cells and the beads were intraperitoneally injected through a 25-gauge syringe needle, cells on GelFoam and alginate were surgically implanted intraperitoneally. In all cases, animals received 5×10⁶ cells except for intravenous injection (2×10⁶ cells/mouse). Weekly 200-μl retro-orbital bleedings were obtained from each implanted NOD/SCID mouse. Two aliquots of 50 μl serum were recovered by centrifugation of the blood samples at 8000 g for 10 min and stored at −80° C. until analyzed. The level of hIL-3 in serum was measured with an hIL-3 enzyme-linked immunosorbent assay (ELISA) kit (BioSource International, Camarillo, Calif.), following the procedure suggested by the manufacturer with the following modifications. Fifty microliters of serum was diluted to 200 μl by addition of diluent buffer and preabsorbed onto C8 MaxiSorp plate (Nunc. Inc.) for 1 h at room temperature to eliminate nonspecific binding. Human IL-3 was determined in triplicate by the transfer of this material to the ELISA plate. ELISA plates were read on a microplate reader (Bio-Rad) and values obtained from a similarly treated standard curve.

For assaying hIL-3 secretion, hMSCS were passaged when cells reached 90% confluence by transferring 0.25 to 0.5×10⁶ hMSCs into a 75-cm flask with 12 ml of hMSC medium. Twenty-four hours later, 1 ml of culture supernatant was collected and stored at −80° C. The assay was performed in triplicate using the hIL-3 ELISA kit. The level of hIL-3 was normalized to the level of endogenously expressed hIL-6 measured with an hIL-6 ELISA kit (BioSource International) using the procedures suggested by the manufacturer. Plates were read on a microplate reader and the data were analyzed using SigmaPlot and Microsoft Excel.

### Results

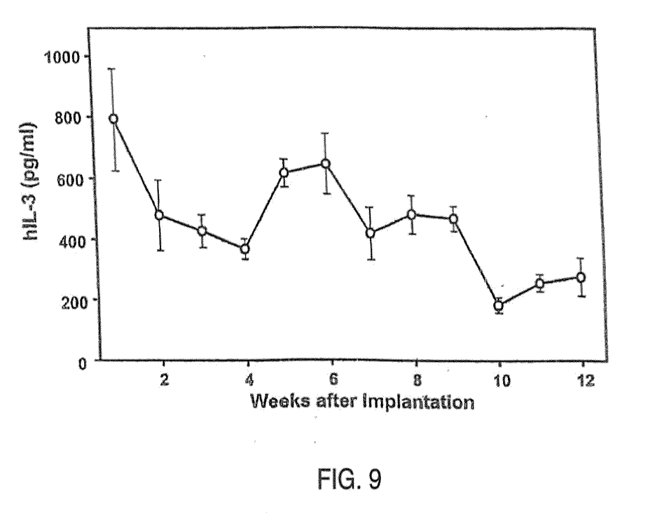

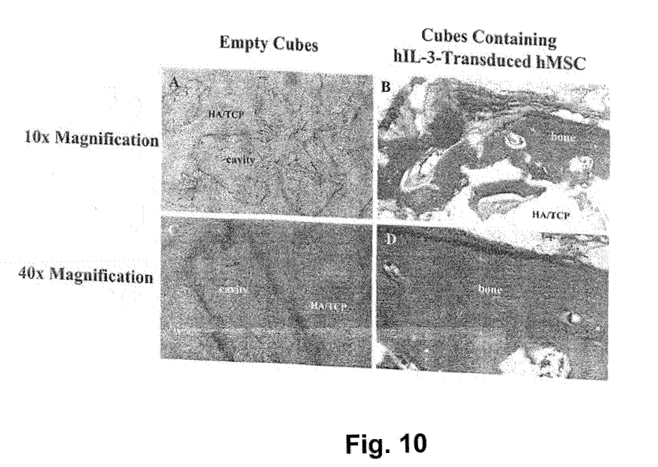

Transgene secretion in vivo. In order to evaluate in vivo expression from transduced hMSCs, hIL-3-transduced hMSCs were implanted into NOD-SCID mice. The hMSCs were transduced with pJM538neo, selected with G418 in culture for 2 weeks, and absorbed on HA/TCP porous ceramic cubes at density of 0.5×10⁶ cells/cube. Cubes (10/animal) were surgically implanted subcutaneously in the lower back of the NOD/SCID mice. Serum levels of hIL-3 produced by the implants were monitored weekly by ELISA. The level of hIL-3 in serum was the highest in the first week after implantation (800±150 pg/ml) and the levels remained in the 200-700 pg/ml range for the remainder of the 12-week time course (FIG. 9). At the end of the 12^{th} week, cubes were removed and placed in culture. After 24 h, supernatants were assayed for hIL-3 protein expression and the cubes were processed for histology. Analysis of the supernatants demonstrated that the cells attached to the cubes were still expressing hIL-3 protein expression and the cubes were processed for histology. Analysis of the supernatants demonstrated that the cells attached to the cubes were still expressing hIL-3 protein (1200±300 pg/cube/24 h). Histology of sections of the cubes with a modified Malloy's aniline blue stain (Sterchi, et al., J. Histotechnol., Vol. 21, pgs. 129-133 (1998)) revealed the presence of mineralized bone within the cube (FIG. 10). These results demonstrated that the transduced hMSCs maintained their stem cell phenotype and transgene expression after 3 months of in vivo implantation in NOD/SCID mice.

In addition to implantation on HA/TCP cubes, hIL-3-transduced hMSCs were tested for systemic detection by other delivery routes. The effect of intravenous and intraperitoneal delivery on hIL-3 on plasma levels was tested. The results are shown in Table 2 below.

TABLE 2
Intravenous and Intraperitoneal Delivery of
hIL-3-Transduced hMSCs to NOD/SCID Mice

| | hIL-3 level is serum (pg/ml)^{a} | | | |
|---|---|---|---|---|
| Route/matrix | 7 days | 14 days | 21 days | 28 days |
| Intravenous/ | 49 ± 20 | 5 ± 5 | 11 ± 11 | 10 ± 7 |
| no matrix | | | | |
| Intraperitoneal/ | 148 ± 30 | 173 ± 37 | 106 ± 42 | 140 ± 56 |
| no matrix | | | | |
| Intraperitoneal/ | 276 ± 25 | 89 ± 38 | 162 ± 24 | 104 ± 24 |
| alginate | | | | |
| Intraperitoneal/ | 440 ± 26 | 166 ± 63 | 168 ± 49 | 257 ± 31 |
| GelFoam | | | | |
| Intraperitoneal/ | 700 ± 54 | 258 ± 35 | 148 ± 51 | 298 ± 18 |
| CultiSpher | | | | |

^{a}Values are means ± standard errors of the mean for serum samples of 2-5 mice in each group. Prebleeds: 27 ± 13 pg/ml of hIL-3, detection limit is 25 pg/ml.

For intravenous injection of hIL-3-transduced hMSCs (2×10⁶ cells), systemic hIL-3 was only slightly above detection in the plasma after 1 week and undetectable thereafter. For intraperitoneal delivery hIL-3-transduced hMSCs (5×10⁶ cells), cells were injected as a cell suspension, adhered to collagen beads, or embedded within a matrix material. Attachment to collagen beads was accomplished by adherence to CultiSpher beads. Two matrixes, alginate and GelFoam, were used to embed hIL-3-transduced hMSCs into the wall of the peritoneal cavity of NOD/SCID mice. The levels of hIL-3 assayed in the serum of animals that received cells intraperitoneally are shown in Table 2. The CultiSpher-attached transduced hMSCs showed the highest level of systemic hIL-3 followed by the GelFoam, alginate, and cells injected without matrix.

The disclosures of all patents, publications (including published patent applications), depository accession numbers, and database accession numbers are hereby incorporated by reference to the same extent that each patent, publication, depository accession number, and database accession number were specifically and individually incorporated by reference.

It is to be understood, however, that the scope of the present invention is not to be limited to the specific embodiments described above. The invention may be practiced other than as particularly described and still be within the scope of the accompanying claims.

1-23. (canceled)

24. A method of treating diabetes in an animal, comprising:
administering intraperitoneally to said animal mesenchymal stem cells genetically engineered with a polynucleotide encoding insulin, said mesenchymal stem cells being administered in an amount effective to treat diabetes in said animal.

25. The method of claim 24 wherein said mesenchymal stem cells are supported on a support.

26. The method of claim 25 wherein said support is a macroporous gelatin bead.

27. The method of claim 24 wherein said animal is a mammal.

28. The method of claim 27 wherein said mammal is a human.

29. The method of claim 24, wherein said mesenchymal stem cells are non-autologous mesenchymal stem cells.

30. The method of claim 24, wherein said mesenchymal stem cells are human mesenchymal stem cells.

31. The method of claim 24, wherein said mesenchymal stem cells are bone marrow-derived.

32. The method of claim 24, further comprising expanding said mesenchymal stem cells in culture.

33. The method of claim 24, wherein said mesenchymal stem cells are expanded in culture for three to eight passages.

34. The method of claim 33, wherein said mesenchymal stem cells are expanded in culture for four to six passages.

35. The method of claim 34, wherein said mesenchymal stem cells are expanded in culture for five passages.

36. The method of claim 24, wherein said mesenchymal stem cells are CD73⁺.

37. The method of claim 24, wherein said mesenchymal stem cells are CD105⁺.

38. The method of claim 24, wherein said mesenchymal stem cells are non-autologous CD73⁺/CD105⁺ mesenchymal stem cells.

39. A method of treating diabetes, comprising:
administering systemically to a patient mesenchymal stem cells genetically engineered to express insulin, said mesenchymal stem cells being administered in an amount effective to treat diabetes in said patient.

40. The method of claim 29, wherein said mesenchymal stem cells are administered intravenously or intraperitoneally.

41. The method of claim 39, wherein said mesenchymal stem cells are non-autologous mesenchymal stem cells.

42. The method of claim 39, wherein said mesenchymal stem cells are human mesenchymal stem cells.

43. The method of claim 39, wherein said mesenchymal stem cells are bone marrow-derived.

44. The method of claim 39, further comprising expanding said mesenchymal stem cells in culture.

45. The method of claim 39, wherein said mesenchymal stem cells are expanded in culture for three to eight passages.

46. The method of claim 45, wherein said mesenchymal stem cells are expanded in culture for four to six passages.

47. The method of claim 46, wherein said mesenchymal stem cells are expanded in culture for five passages.

48. The method of claim 39, wherein said mesenchymal stem cells are CD73⁺.

49. The method of claim 39, wherein said mesenchymal stem cells are CD105⁺.

50. The method of claim 39, wherein said mesenchymal stem cells are non-autologous CD73⁺/CD105⁺ mesenchymal stem cells.
